(19) United States
(12) Patent Application Publication
(10) Pub. No.: US 2010/0260731 A1
(43) Pub. Date: Oct. 14, 2010

(54) PROPAGATION OF PRIMARY CELLS AND THE USE THEREOF

(76) Inventors: Adrianus J.C.M. Braspenning, Schonbrunn-Haag (DE); Stefan Holder, Stuttgart (DE); Heiner Kupper, Goch (DE)

Correspondence Address: WENDEROTH, LIND & PONACK, L.L.P., 1030 15th Street, N.W.,, Suite 400 East, Washington, DC, 20005-1503, US

(21) Appl. No.: 12/733,441

(22) PCT Filed: Sep. 3, 2008
(86) PCT No.: PCT/DE2008/001470

§ 371 Date: Apr. 14, 2010

(30) Foreign Application Priority Data
Sep. 3, 2007 (DE) 10 2007 041 655.7

(51) Int. Cl. C12N 15/85; C12N 15/86; C12Q 1/02; C40B 40/02; A01N 1/00; A61K 35/12; C12Q 1/68

(52) U.S. Cl. 424/93.21; 435/455; 435/366; 435/456; 435/29; 506/14; 435/1.1; 424/93.7; 435/6

(57) ABSTRACT

The invention relates to a method for propagating or concentrating primary cells without tumorous characteristics and to the subsequent use thereof.

The invention relates to a method for propagating or enriching primary cells without timorous characteristics, and to the subsequent use thereof.

Providing cells that are suitable for humans presents a medical challenge, whether these cells are intended for therapeutic in-vivo use or for use in the development of in-vitro cell systems including corresponding cell cultures. Moreover, there is an urgent need to establish cell cultures that are as close as possible to human cells, which can then be used for pharmaceuticals testing, research, etc.

In the prior art, cell lines have been established for cells that are able to propagate indefinitely on the proper culture media and are immortal. In particular, tumor cells or tumor-like cells are known, along with the previously known HeLa cells—cervical carcinoma cell line, COS cells, HEK-293 kidney cells, Chinese hamster ovary (CHO) cells, HEp-2—human epithelial laryngeal carcinoma cell line, etc. The production of such cell lines is described in EP833934 (Crucell), for example. Such cell lines are used in pharmaceuticals testing, for example. The drawbacks of such cell lines, however, are the genetic mutations (such as point mutations, exchanges of chromosome pieces (rearrangements), increased numbers of gene copies (gene amplification) and even changes to sets of chromosomes (aneuploidy)), and the tumorous characteristics that result from immortalization and an unlimited capacity to divide. The cells of such cell lines are known to gradually change through spontaneous mutations during the course of cultivation; they can develop to a malignant cell population and are genetically unstable. According to the findings of the inventor, in such cases a critical threshold of cumulative mutations occurs in the culture after only approximately 60 cell divisions. Such mutations could lead to the activation of oncogenes or to the inactivation of tumor-suppressor genes. Thus a cell population can be infiltrated by cells that have increased proliferative activity as a result of the cumulative mutations. This selection process corresponds to the precancerous stage of tumor formation; in addition, most commercially available cell lines have already undergone an unknown number of doublings, and may even themselves be derived from malignant tumor cells.

TABLE 1
Characteristics of cells with expanded doubling
capacity as compared with other cell systems

| | | Cells with | |
|---|---|---|---|
| | | Expanded | |
| Cell System | Primary | Doubling | Tumor Cell |
| Characteristic | Cells | Capacity | Lines |
| Production | from natural | from primary | from tumors |
| | tissues | cells | |
| Number of possible | Limited to | Limited to | Unlimited |
| cell divisions | 10-25^{a} | 30-60 | |
| Phenotype | Natural | Natural | Altered |
| Genetically stable | Yes | Yes | No |
| Growth in soft | No | No | Yes |
| Agar^{b} | | | |
| Tumor growth in | No | No | Yes |
| vivo^{c} | | | |

^{a}The actual proliferative capacity of cells is dependent upon the age of the cell donor; the older the donor, the more divisions the cells have undergone.
^{b}The cells to be studied are coated with a special agarose (soft agar); cells that have lost their natural capacity for contact inhibition grow as colonies into the soft agar. This is also often referred to as transformation and is a precondition for tumor formation. However, in most cases tumor formation requires additional stages, such as cell immortalization, for example. Natural cells are unable to grow in soft agar.
^{c}As a rule, the soft agar test does not provide sufficient proof of malignant cell degeneration. One frequently used test that is known to one of ordinary skill in the art involves grafting the cells to be tested in immunodeficient mice (nude mice or SCID mice) . Due to the deficient or absent immune system, even cells of other species (xenografts) are not rejected immunologically, and malignant tumor cells are able to develop into tumors.

However, there is an urgent need to obtain cells from primary cell cultures, which have an expanded natural capacity for proliferation as compared with primary cells, but which optimally have none of the characteristics of tumor cells, in particular those of malignant tumor cells, such as growth in soft agar or even tumor growth in vivo, for example, and which for the most part have accumulated no mutations.

The availability of primary cells is of great importance not only for research and for the biotechnology and pharmaceuticals industries, but also for cell-based therapies for the treatment of degenerative diseases. Such diseases include, for example, myocardial insufficiency, cirrhosis of the liver, Parkinson's disease and insulin-dependent diabetes.

However, the shortage of primary cells has heretofore limited their use.

Within the scope of the invention, the term “primary cells” refers to explants obtained directly from bodily fluids or bodily tissues of multicellular organisms, such as humans, for instance, and having normal, i.e., not degenerated, cells. Primary cell cultures are primary cells that have been cultured up to the first passage. Primary cells have natural differentiation characteristics and are mortal.

Within the scope of the invention, the term “type I cells” refers to those primary cells which can be propagated in culture, but which cease to grow and die off after a small number of population doublings. Type I cells constitute a small number of primary cell types. The mortality of these cells severely limits their commercial use. Examples of such type I cells include endothelial cells (vascular cells), keratinocytes (skin cells), and fibroblasts (connective tissue cells). Within the scope of the invention, the term “type II cells” refers to those primary cells whose proliferative capacity in the culture is arrested from the very start, and which can therefore not be brought to propagation. Type II cells constitute the vast majority of primary cells of multicellular organisms, such as humans, for example. Examples of such type II cells include cardiomyocytes (cardiac muscle cells), islet cells (insulin-producing cells of the pancreas), neurons (nerve cells), etc.

It has long been known that the proliferative capacity of primary cell cultures is limited. As early as 1961, Leonard Hayflick of the Wistar Institute (US) discovered that fibroblasts of neonatal infants are able to undergo 80-90 divisions, while those of 70-year-olds replicate only 20-30 times (summary in: HAYFLICK, LEONARD The biology of human aging. American Journal of the Medical Sciences. 265(6):432-446, June 1973). After these numbers of divisions, cells enter into senescence. Also described for primary cells is the so-called extended lifespan, in which the proliferative capacity of primary cells is increased for the purpose of studying carcinogenesis resulting from infiltration by viral oncogenes such as SV40 TAg, adenovirus E1A, HPV E6 and E7, or cellular oncogenes such as c-ras and c-myc. In the systems that were studied, efforts were made to immortalize the cells beyond their “extended lifespan” through selection, mutagenesis and other measures, to allow the effects of the viral or cellular gene on carcinogenesis to be studied.

To propagate cells beyond their extended lifespan, a process must be used which compensates for the shortening of the chromosomal telomeres that occurs with each cell division. One such possible process involves the use of telomerase (Harley, C. B. and B. Villeponteau. 1995. Telomeres and telomerase in aging and cancer. Curr. Opin. Genet. Dev. 5:249-255). Cells which are able to compensate for telomere loss using telomerase, for example, have unlimited proliferative capacity, or immortality. Disadvantageously, however, during the course of cell division mutations unavoidably occur, which sooner or later must result in carcinogenesis.

Not known in the prior art, however, is a targeted enrichment or propagation—including extraction—of primary cells in which the development of tumor cell characteristics, such as growth in soft agar or tumor growth in vivo, is prevented.

The object of the invention is therefore to provide such a method for enriching or propagating primary cells that, according to the method, are optimally without tumorous characteristics.

The object is attained with a method for propagating primary cells, wherein in the following steps human primary cells
a.) are isolated,
b1.) with at least one proliferation gene or its gene product being functionally introduced into the cell and/or
b2.) at least one cellular factor that induces an arrest of cell division being inactivated,
c.) the cells are cultivated and/or passaged and
d.) are harvested, wherein the harvested cells have no tumorous characteristics.

Preferably more than three additional passages, more than five additional passages, preferably 20-40 additional passages can be performed as compared with untreated primary cells. By limiting cell division to 20-40 additional doublings, the undesirable mutations, which necessarily occur in immortalized cells after more than 60 doublings, are eliminated.

The invention therefore relates to a method of this type comprising the steps a.)-d.), wherein in step c.) up to 20-40 passages can be achieved without the resulting cells having tumorous characteristics.

Particular advantageously, therefore, the method of the invention ensures that the resulting cells will take on none of the characteristics of tumor cells, especially of malignant tumor cells, such as growth in soft agar and tumor growth in vivo, for example (growth of tumors in xenograft animal models). The term “tumorous characteristic” does not, however, refer to the expanded doubling capacity of the target cells resulting from the method of the invention.

Also advantageously, the method of the invention does not produce immortalization of the resulting cells. The method of the invention thus permits the enrichment or propagation of resulting non-immortalized cells.

Cultivation is performed using culture media that are known to one of ordinary skill in the art.

The method of the invention permits the advantageous enrichment and propagation of primary cells with expanded doubling capacity, which are also essentially genetically unaltered, like primary cells following cultivation, whereas most cell lines contain many genetic alterations.

The term “harvest” as used in the invention means that the cells that are obtained can be continuously or discontinuously removed or extracted from the propagation, and subsequently applied and used in any units (quantity, quality, etc.).

The term “propagation or enrichment of primary cells” means the preparation of “cells with expanded doubling capacity” (see comparative Table 1), wherein process feature b1.) or b2.) of claim 1 leads to a structural alteration of primary cells of the parent material.

The expanded doubling capacity advantageously permits the generation of a substantially increased quantity of cells.

Within the scope of this invention, a proliferation gene is one which improves cell division and enables a limited expansion of cell proliferation capacity in the primary cell, wherein the probability of cell transformation or alterations to the differentiation properties is very significantly reduced as compared with cell lines of the prior art. In particular, the proliferation gene is not an immortalizing gene. According to the invention, the proliferation gene is preferably chosen from the group of viral proliferation genes: E6 and E7 of papillomaviruses such as HPV (human papillomavirus) and BPV (bovine papillomavirus), for example; the large and small TAg of polyomaviruses, such as SV40, JK-virus and BC-virus, for example; the E1A and E1B adenoviral proteins, EBNA proteins of the Epstein-Barr virus (EBV); and the proliferation gene of HTLV and herpesvirus saimiri and their respective coding proteins, or is chosen from the group of cellular proliferation genes, especially the following classes of genes: myc, jun, ras, src, fyg, myb, E2F and Mdm2 and TERT (catalytic subunit of telomerase), preferably the human telomerase (hTERT). In a further embodiment, the transforming activity of the aforementioned TAg (1-708 AS, e.g., SV40) in the area of amino acids 1-121 and/or 137 to 708 is eliminated by means of point mutations, deletions and/or insertions, while maintaining the p53 binding domain (“bipartite domain”) (Ruppert, Stilman (1993), Analysis of a protein binding domain p53, Mol Cell. Biol. 13, 3811-3820).

According to the invention, however, viral proliferation genes are preferable, with E6 and E7 of HPV or BPV being particularly preferred. HPV type proliferation genes which are associated with malignant diseases can also be used. The best known examples of “high-risk” papillomaviruses are HPV16 and HPV18. Additional examples from the high-risk group include HPV 31, 33, 35, 39, 45, 51, 52, 56, 58, 59, 68, 73 and 82. However, the proliferation genes E6 and E7 of so-called “low-risk” HPV's may also be used. Known examples include HPV types 6 and 11; additional HPV types from the low-risk group include HPV 40, 42, 43, 44, 54, 61, 70, 72 and 81.

The importance of E6 proteins in connection with an increase in proliferation involves especially the inactivation of the p53 pathway and the induction of telomerase. The importance of E7 proteins in connection with an increase in proliferation involves especially the inactivation of the pRb pathway. Within the context of the invention, the proliferation genes of different serotypes of one virus species or of different virus species can also be combined, or chimeric proliferation genes of different serotypes of one virus species or of different virus species can even be produced and used. For example, one E6 domain of a chimeric gene can be derived from HPV 16, for example, and another from HPV 6. Of course, the proliferation genes can also be truncated or can have one or more base exchanges without departing from the scope of the invention. The aforementioned proliferation genes represent preferred embodiments and are not intended to limit the invention. The proliferation gene can also be the subject of a synthetic or artificially produced gene sequence.

These factors are “functionally introduced” into the target cells whose cell dividing capacity will be expanded, in which process the following gene transfer systems may be used, among others: transfer of expression constructs of the aforementioned gene functions into cells via the classic calcium-phosphate method (Wigler, M. et al., 1977. Cell 11:223-232), via lipofection (Felgner, P. L. et al, 1987. Proc. Natl. Acad. Sci. U.S.A. 84:7413-7417), via electroporation (Wolf, H. et al., 1994. Biophys. J. 66:524-531), via microinjection (Diacumakos, E. G. 1973. Methods Cell Biol. 7:287-311), via conjugates, which are taken up through cellular receptors or are receptor independent. The aforementioned gene functions can also be transferred to target cells using viral vectors. Examples include retroviral vectors, AAV vectors, adenoviral vectors and HSV vectors, to name just a few examples of vectors (overview of viral vectors in: Lundstrom, K. 2004. Technol. Cancer Res. Treat. 3:467-477; Robbins, P. D. and S. C. Ghivizzani. 1998. Pharmacol. Ther. 80:35-47). The term “functionally introduced” encompasses, in particular, the transfection of the target cells using at least one proliferation gene.

The expression of the aforementioned viral or cellular proliferation genes can be controlled by strong or weak constitutive promoters, by tissue-specific promoters, by inducible promoters (Meyer-Ficca, M. L. et al. 2004. Anal. Biochem. 334:9-19), or the expression cassettes can be flanked by specific sequences for molecular excision systems. Examples include the Cre/Lox system (U.S. Pat. No. 4,959,317), the use of which results in the molecular removal of the expression constructs from the genome of the target cells.

In a further embodiment, the gene products of the proliferation genes can also be functionally introduced into the target cell directly as such, or by means of a fusion protein. The latter are preferably messenger proteins, such as VP22, HIV TAT (Suzuki et al., 277 J. Biol. Chem. 2437-2443 2002 and Futaki 245 Int. J. Pharmaceut. 1-7 (2002), (HIV) REV, Antennapedia polypeptide (WO97/12912 and WO99/11809) or Penetratin (Derossi et al., 8 Trends Cell Biol., 84-87 (1998), engrailed (Gherbassi, D. & Simon, H. H. J. Neural Transm. Suppl 47-55 (2006), Morgan, R. 580 FEBS Lett., 2531-2533 (2006), Han, K. et al. 10 Mol. Cells 728-732 (2000)), or HOXA5 (Chatelin et al. 55 Mech. Dev. 111-117 (1996)), a polymer of L-arginine or D-arginine amino acid residues (Can. Patent No. 2,094,658; U.S. Pat. No. 4,701,521; WO98/52614), a polymer of L-lysine or D-lysine amino acid residues (Mai et al., 277 J. Biol. Chem. 30208-30218 (2002), Park et al. 13 Mol. Cells 202-208 (2002), Mi et al. 2 Mol. Ther. 339-347 (2000)), transcription factors such as BETA2/neuro D, PDX-1 (Noguchi and Matsumoto 60 Acta Med. Okayama 1-11, (2006), Noguchi et al. 52 Diabetes 1732-1737 (2003), Noguchi et al. 332 Biochem. Biophys. Res. Commun. 68-74 (2005)), nuclear localization signal, (Yoneda et al. 201 Exp. Cell Res. 313-320 (1992)), histone-derived peptides (Lundberg and Johansson 291 Biochem. Biophys. Res. Comm. 367-371 (2002)), a polymer of cationic macromolecules, FGF-1 and FGF-2, lactoferrin, i.a., as described previously, in the literature.

Within the scope of this invention, “at least one cellular factor that induces an arrest of cell division is inactivated” means that an arrest of cell division is activated during the course of the senescence program, for example (summary in: Ben Porath, I. and R. A. Weinberg. 2005. Int. J. Biochem. Cell Biol. 37:961-976.), or means the arrest of cell division which is activated in cells within the scope of the differentiation program. For example, it is known that cardiomyocytes lose their proliferative capacity soon after birth, a process which is regulated, i.a., by the expression of cell cycle inhibitors such as p16, p21, p27 (Brooks, G., et al. 1998. Cardiovasc. Res. 39, 301-311; Flink, I. L. et al., 1998. J. Mol. Cell Cardiol. 30, 563-578; Walsh, K. and Perlman, H. 1997. Curr. Opin. Genet. Dev. 7, 597-602). Similar processes undoubtedly apply to the majority of all primary cell types. Thus an elimination of cell cycle inhibitors in differentiated cells could cause the cells to reenter the proliferation phase. Within the context of the invention, this also applies to other cell cycle-inhibiting proteins not mentioned here.

Within the scope of the invention, the p53 protein, which is important to regulating the cell cycle, and all proteins that bind directly to p53, upstream, and/or downstream factors of this p53 pathway can be generally eliminated, in order to achieve the goal of expanded cell proliferation capacity (overview of the p53 pathway in: Giono, L. E. and J. J. Manfredi. 2006. J. Cell Physiol 209: 13-20; Farid, N. R. 2004. Cancer Treat. Res. 122:149-164).

Within the scope of the invention, the p16/INK4a protein, which is important to regulating the cell cycle, and all proteins that bind directly to p16/INK4a, upstream, and/or downstream factors of this p16 pathway can be generally eliminated, in order to achieve the goal of expanded cell proliferation capacity (overview of the p16/INK4a pathway in: Shapiro, G. I. et al., 2000. Cell Biochem. Biophys. 33:189-197).

Within the scope of the invention, the pRb protein, which is important to regulating the cell cycle, and/or the other members of the pRb family (e.g., p107, p130), and all proteins that bind directly to members of the pRb family, upstream, and/or downstream factors of this pRb pathway can be generally eliminated in order to achieve the goal of expanded cell proliferation capacity (overview of the pRb pathway in: Godefroy, N. et al. 2006. Apoptosis. 11:659-661; Seville, L. L. et al. 2005. Curr. Cancer Drug Targets. 5:159-170).

The inactivation of cellular factors such as p53, pRb, p16, etc. can be achieved via the expression of dominant negative mutants of the corresponding factors, for example (Herskowitz, I. 1987. Nature 329:219-222; Küpper, J. H., et al. 1995. Biochimie 77:450-455), via inhibition of the gene expression of these factors using antisense oligonucleotides (Zon, G. 1990. Ann. N.Y. Acad. Sci. 616:161-172), RNAi molecules (Aagaard, L. and J. J. Rossi. 2007. Adv. Drug Deliv. Rev. 59:75-86; Chakraborty, C. 2007. Curr. Drug Targets. 8:469-482), morpholinos (Angerer, L. M. and R. C. Angerer. 2004. Methods Cell Biol. 74:699-711), ribozymes (Sioud, M. and P. O. Iversen. 2005. Curr. Drug Targets. 6:647-653), or via gene knockout (Le, Y. and B. Sauer. 2000. Methods Mol. Biol. 136:477-485; Yamamura, K. 1999. Prog. Exp. Tumor Res. 35:13-24). These methods are known to one of. ordinary skill in the art and are described extensively in the literature. Inactivation can also be achieved through the action of specific antibodies (e.g., single chain antibodies, intrabodies, etc.; overview in: Leath, C. A., III, et al. 2004. Int. J. Oncol. 24:765-771; Stocks, M. R. 2004. Drug Discov. Today 9:960-966). Inactivation can also be achieved using chemical inhibitors of the cellular factors, for example using kinase inhibitors.

One example of a kinase inhibitor is the substance Imatinib (Glivec®). With this substance, a reduction in cell proliferation is achieved. Imatinib is a specific inhibitor which blocks the activity of the tyrosine kinase ABL in diseased cells, thereby suppressing a pathologic increase in the propagation of mutated blood stem cells.

These cells with expanded doubling capacity obtained according to the method are classified between primary cells and immortalized cell lines: Cells with expanded doubling capacity have most of the natural characteristics of primary cells, and can advantageously undergo significantly more cell divisions.

The advantages are the following:

Cells with expanded doubling capacity can be universally generated from all type I and type II cells. Cells with expanded doubling capacity are suitable for use in basic biomedical research, for the development and testing of medications, cosmetics, foods and textile additives.

A further advantage is their simple and cost-effective handling in the cell culture, i.e., no costly medium additives are necessary.

Example illustrating the expanded doubling capacity of the harvested cells from the method of the invention:

Starter culture comprised of 1000 primary endothelial cells having a replicative capacity of 15 doublings:

Yield of available cells=2E15×1000=3.3×10E7 cells

Starter culture of 1000 “cells with expanded doubling capacity,” having a replicative capacity of 40 doublings:

Yield of available cells=2E40×1000=1.1×10E15 cells, in other words eight powers of ten more.

Based upon these advantageous characteristics of the harvested “cells with expanded doubling capacity” obtained according to the invention, subsequent processes and applications of such harvested cells can be advantageously applied.

The invention therefore also relates in one preferred embodiment to a method for producing an assay, comprising the following steps:
a.) preparing a substrate material,
b.) immobilizing or fixing at least one harvested cell according to the method of the invention on said substrate material, and
placing this cell from b.) in contact with an analyte.

The invention further relates to the use of the obtained cells according to the method of the invention to perform an assay, wherein such “cells with expanded doubling capacity” are mixed with at least one analyte chosen from the group of chemical substances (e.g., synthetic or native substances and mixtures thereof), medications, active substances, cosmetics, cells.

Such analytes may have any targets, for example, DNA, RNA, proteins, lipids, sugar, etc. In the case of a cell assay, often the activity of one or more targets is measured, for example the reaction of one or more enzymes, the transport of a substance through a biological membrane, the binding of a ligand to a receptor, or more broadly, the replication of DNA, cell division or cell death, to name just a few examples.

Proof of a positive event can be provided in the broadest sense using an analytical reagent, e.g., using a fluorescence-marked antibody or the like. This would include, in particular, suitable bioanalytical processes, such as immunohistochemistry, antibody arrays, luminex/luminol, ELISA, immunofluorescence, radioimmunoassays, for example.

The invention further relates to a cell bank containing harvested cells from the method of the invention, in other words such “cells with expanded doubling capacity,” which are in suspension or can be placed on a solid substrate.

The term “solid substrate” comprises embodiments such as a filter, a membrane, a magnetic bead, a silicon wafer, glass, plastic, metal, a chip, a mass spectrometry target or a matrix comprised of proteins, for example, or other matrices, such as PEG, etc., for example.

In a further preferred embodiment of the arrangement of the invention (synonym: array), said array corresponds to a lattice on the order of magnitude of a microtiter plate (96 wells, 384 wells, or more), a silicon wafer, a chip, a mass spectrometry target or a matrix.

The substrate material (matrix) can be in the form of spherical, unaggregated particles, so-called beads, fibers or a membrane, wherein a porosity of the matrix increases the surface. Porosity can be achieved, for example, in a customary manner by adding pore formers, such as cyclohexanol or 1-dodecanol, to the reaction mixture of the suspension polymerization.

The invention further relates to a pharmaceutical substance containing harvested cells from the method of the invention for the treatment of diseases, especially cardiac insufficiency, cirrhosis of the liver, Parkinson's disease, and insulin-dependent diabetes. With these degenerative diseases, the propagated cells are obtained from a primary cell from the patient's own body. The harvested cells are returned to the patient (e.g., by injecting heart cells into the heart muscle, etc.).

In a preferred embodiment, E6 and E7 of the low-risk HPV are used as viral proliferation genes.

The invention further relates to a starter culture containing harvested cells from the method of the invention, in other words harvested cells comprised of “cells with expanded doubling capacity” and customary additives and auxiliary agents.

In a further embodiment of the invention, the harvested cells of the method of the invention are used for a culture on customary media, especially as co-cultivating cells (feeder cells), for enriching target cells (e.g., stem cells, etc.).

In a further embodiment of the invention, the harvested cells of the method of the invention are used to produce 3D cell models or in-vitro tissue, including as models for human skin and other organs (heart, liver, etc.), bones, cartilage, optionally applied to a substrate (so-called scaffold biomaterials).

1. Method for propagating or enriching primary cells, comprising the steps wherein human primary cells
a.) are isolated,
b1.) are functionally introduced into the cell with at least one proliferation gene or its gene product and/or
b2.) at least one cellular factor, which induces an arrest of cell division, is inactivated,
c.) the cells are cultivated and/or passaged and
d.) are harvested, wherein the harvested cells have no tumorous characteristics.

2. Method of claim 1 for propagating or enriching primary cells, wherein in step c.) 20 to 40 passages occur.

3. Method of claim 1 for propagating or enriching primary cells, wherein the harvested cells in step d.) are no longer able to grow in soft agar or exhibit tumor growth in vivo.

4. Method of claim 1 for propagating or enriching primary cells, wherein the obtained cells are not immortalized.

5. Method of claim 1 for propagating or enriching primary cells, characterized in that the proliferation gene in b1.) is a cellular and/or viral proliferation gene.

6. Method of claim 1 for propagating or enriching primary cells, characterized in that the cellular proliferation gene is chosen from the group myc, jun, ras, src, fyg, myb, E2F and Mdm2 and TERT, or the viral proliferation gene is chosen from the group E6 and E7 of papillomaviruses such as HPV, for example; the large and small TAg of polyomaviruses such as SV40, JK virus and BC virus, for example; the E1A and E1B adenoviral proteins, EBNA proteins from the Epstein-Barr virus (EBV); and HTLV and herpesvirus saimiri.

7. Method of claim 1 for propagating or enriching primary cells, characterized in that the cellular factor in b.) is chosen from the group p53, p16, pRb, p107, p130 or the respective upstream or downstream factors thereof or proteins that bind thereto in the pathway, and the inactivation of such cellular factors is achieved via the expression of dominant negative mutants or via inhibition of the gene expression of these factors using antisense oligonucleotides, RNAi molecules, morpholinos, ribozymes, or via gene knockout, via the action of specific antibodies, chemical inhibitors.

8. Method of claim 1 for propagating or enriching primary cells, characterized in that the viral proliferation genes are E6 and E7 of HPV or BPV, especially HPV16 and HPV18 and HPV 31, 33, 35, 39, 45, 51, 52, 56, 58, 59, 68, 73 and 82 and/or HPV6 and HPV 11, and HPV 40, 42, 43, 44, 54, 61, 70, 72, and 81.

9. Method for producing an assay, comprising the following steps:
a.) preparing a substrate material,
b.) immobilizing or fixing at least one harvested cell of claim 1 on said substrate material and
placing this cell from b.) in contact with an analyte.

10. Method of claim 9 for producing an assay, characterized in that the analyte is chosen from the group comprised of chemical substances, medications, active substances, cosmetics, cells.

11. Cell bank containing harvested cells according to claim 1 on a solid substrate or in suspension.

12. 3D cell model or in-vitro tissue containing harvested cells of claim 1, optionally on a solid substrate.

13. Starter culture, co-cultivating cells, or culture containing harvested cells of claim 1, along with customary additives and auxiliary agents.

14. Pharmaceutical substance containing harvested cells of claim 1, especially for the treatment of diseases, in particular degenerative diseases such as cardiac insufficiency, cirrhosis of the liver, Parkinson's disease, and insulin-dependent diabetes, along with customary additives and excipients.

15. Use of the harvested cells according to a method of claim 1 for producing a cell bank, 3D cell model or in-vitro tissue, starter culture, culture, co-cultivating cells, pharmaceutical substance.
